(19) United States
(12) Patent Application Publication
(10) Pub. No.: US 2026/0124302 A1
(43) Pub. Date: May 7, 2026

(54) CAR-T CELL WITH GOOD BLOOD-BRAIN BARRIER PERMEABILITY, PRODUCT, AND USE THEREOF

(71) Applicant: TONGJI HOSPITAL,TONGJI MEDICAL COLLEGE,HUAZHONG UNIVERSITY OF SCIENCE AND TECHNOLOGY, Wuhan (CN)

(72) Inventors: Daishi TIAN, Wuhan (CN); Chuan QIN, Wuhan (CN); Wei WANG, Wuhan (CN); Xiaowei PANG, Wuhan (CN); Luoqi ZHOU, Wuhan (CN); Minghao DONG, Wuhan (CN); Yang YU, Wuhan (CN); Lulu XU, Wuhan (CN); Yuxin LIU, Wuhan (CN)

(73) Assignee: TONGJI HOSPITAL,TONGJI MEDICAL COLLEGE,HUAZHONG UNIVERSITY OF SCIENCE AND TECHNOLOGY, Wuhan (CN)

(21) Appl. No.: 19/379,754

(22) Filed: Nov. 5, 2025

Related U.S. Application Data
(63) Continuation of application No. PCT/CN2025/073875, filed on Jan. 22, 2025.

(30) Foreign Application Priority Data
Apr. 15, 2024 (CN) 202410448173.3

(51) Int. Cl. A61K 40/11; A61K 40/31; A61K 40/35; A61K 40/41; A61P 25/00; C12Q 1/6876

(52) U.S. Cl. CPC A61K 40/11; A61K 40/31; A61K 40/35; A61K 40/414; A61P 25/00; C12Q 1/6876; C12Q 2600/158

(57) ABSTRACT

Disclosed is a CAR-T cell with good blood-brain barrier permeability, a product, and use thereof, relating to the technical field of treatment of central nervous system diseases. Based on the study on CAR-T cell immunotherapy for central nervous system diseases, this disclosure provides a CAR-T cell with good blood-brain barrier permeability and a corresponding drug for treating or improving the central nervous system diseases. The CAR-T cell targets B-cell maturation antigens and highly expresses a chemokine receptor CXCR3 and chemokines CCL1, CCL3, and CCL4. This disclosure further provides a product for detecting and judging the blood-brain barrier permeability of the CAR-T cell, such as probes, reagents, and kits, which can accurately detect and judge the blood-brain barrier permeability of a specific CAR-T cell.

### TECHNICAL FIELD

This disclosure relates to the technical field of treatment of central nervous system diseases, and in particular, to a CAR-T cell with good blood-brain barrier permeability and use in detecting the blood-brain barrier permeability of the CAR-T cell for treating the central nervous system diseases.

### SEQUENCE LISTING

The sequence listing xml file submitted herewith, named “WI-US25-31222-P.xml”, created on Oct. 29, 2025, and having a file size of 6,144 bytes, is incorporated by reference herein.

### BACKGROUND

Chimeric antigen receptor T-cell (CAR-T) immunotherapy is a type of immunotherapy. CAR-T cell immunotherapy is implemented by collecting and isolating T cells from a patient's blood, and then culturing and amplifying them in large quantities in vitro after genetically modifying them, and finally infusing them back into the patient's body to eliminate target cells in a targeted manner, thereby playing its unique therapeutic effect. At present, the CAR-T cell immunotherapy is mainly used for the clinical treatment of patients with hematological malignancies and malignant tumors, such as relapsed/refractory B-cell acute lymphoblastic leukemia, large B-cell non-Hodgkin lymphoma un-responded to conventional treatment, relapsed/refractory malignant lymphoma, and relapsed/refractory multiple myeloma, and shows remarkable curative effect and sustained persistence to these diseases. Due to its capability of directly inducing target cell death and self-amplification characteristics, the CAR-T cell immunotherapy has been recommended as the second-line treatment for B-cell lymphoma within one year after initial treatment.

However, although the CAR-T cell immunotherapy has showed therapeutic effectiveness in many types of solid tumors and hematological tumors, due to its special structure of blood-brain barrier in the central nervous system (CNS), the CAR-T cell immunotherapy has not achieved satisfactory results in the treatment of CNS diseases. The blood-brain barrier is a dynamic barrier between the CNS and a blood circulation system, which strictly regulates the transport of substances between blood and brain/cerebrospinal fluid, provides a stable internal environment for the realization of a neurological function, protects the CNS from various harmful substances, and also becomes a barrier that drugs for treating CNS-related diseases have to face. Many drugs have been eliminated because they cannot cross the blood-brain barrier to reach an effective dose. In the treatment of CNS diseases, including central nervous system leukemia, brain tumors such as glioblastoma, encephalitis, and neuromyelitis optica spectrum disorders, there are high requirements for the capability of drugs to effectively cross the blood-brain barrier.

In the process of the CAR-T cell immunotherapy of the central nervous system diseases, whether the CAR-T cell can effectively cross the blood-brain barrier to enter the central nervous system and play its targeted therapeutic effect is very important. Therefore, it is urgent to develop a reliable CAR-T cell with good blood-brain barrier permeability in the treatment of CNS diseases.

### SUMMARY

This disclosure provides a CAR-T cell for treatment of central nervous system diseases and having good blood-brain barrier permeability, a product in detecting the blood-brain barrier permeability of the CAR-T cell, and corresponding use thereof. The objectives are achieved by the following technical solutions.

Provided is a CAR-T cell with good blood-brain barrier permeability, where the CAR-T cell is used for treating or improving central nervous system diseases, and the CAR-T cell targets B-cell maturation antigens and highly expresses a chemokine receptor CXCR3 and chemokines CCL1, CCL3, and CCL4.

This disclosure confirms that the CAR-T cell can effectively cross the blood-brain barrier. The CAR-T cell has good chemotaxis and a capability of activating chemotaxis-related pathways.

Furthermore, the CAR-T cell is derived from peripheral blood mononuclear cells (PBMCs) isolated from peripheral blood.

This disclosure further provides a method for detecting the blood-brain barrier permeability of the aforementioned CAR-T cell that is not aimed at disease detection and diagnosis. Judgment is made by detecting expression levels of the chemokine receptor CXCR3 and the chemokines CCL1, CCL3, and CCL4 in cerebrospinal fluid, or by detecting and analyzing the chemotaxis of the CAR-T cell based on single-cell transcriptome sequencing data (for example, obtained by using the 10×genomics platform).

A product for detecting the blood-brain barrier permeability of the aforementioned CAR-T cell is provided. The CAR-T cell is used for treating or improving central nervous system diseases, and the product is used for detecting the expression levels of the chemokine receptor CXCR3 and the chemokines CCL1, CCL3, and CCL4 in cerebrospinal fluid.

Further, the product includes a reagent or probe for detecting the expression levels of the chemokine receptor CXCR3 and the chemokines CCL1, CCL3, and CCL4.

Furthermore, the product is a detection kit, a test strip, or a detection chip.

This disclosure further provides use of the aforementioned CAR-T cell with good blood-brain barrier permeability, used for preparing drugs for targeted treatment of central nervous system diseases.

Further, the central nervous system diseases are neuromyelitis optica spectrum disorders.

Compared with the prior art, this disclosure has the following beneficial effects: it provides a method for detecting the blood-brain barrier permeability of the CAR-T cell, with high accuracy of detection.

This disclosure further constructs a CAR-T cell product for high expression of the chemokine receptor CXCR3 and the chemokines CCL1, CCL3, and CCL4, and proves its function of effectively crossing the blood-brain barrier. The CAR-T cell has good chemotaxis and a capability of activating chemotaxis-related pathways.

Therefore, the CAR-T cell prepared by the method provided in this disclosure has good blood-brain barrier permeability, which is beneficial for the CAR-T cell to play a more efficient role in the treatment of the central nervous system diseases, especially the neuromyelitis optica spectrum disorders (NMOSDs).

### BRIEF DESCRIPTION OF THE DRAWINGS

FIG. 1 is a graph showing a CAR copy number in cerebrospinal fluid at different time after CAR-T treatment;

FIG. 2 shows expression levels of chemokines and receptors of CAR-T cells effectively crossing blood-brain barrier in CD4+ T cells;

FIG. 3 shows expression levels of chemokines and receptors of CAR-T cells effectively crossing blood-brain barrier in CD8+ T cells;

FIG. 4 is a screening chart of cell markers clusters three months after CAR-T treatment;

FIG. 5 shows proportions of cell clusters three months after CAR-T treatment;

FIG. 6 shows a difference of chemotaxis scores of CAR-T cells in peripheral blood and cerebrospinal fluid;

FIG. 7 shows a difference of levels of chemokine and receptor expression of CAR-T cells in peripheral blood and cerebrospinal fluid;

FIG. 8 is an enrichment diagram of chemotaxis-related up-regulated pathways of CAR-T cells in cerebrospinal fluid; and

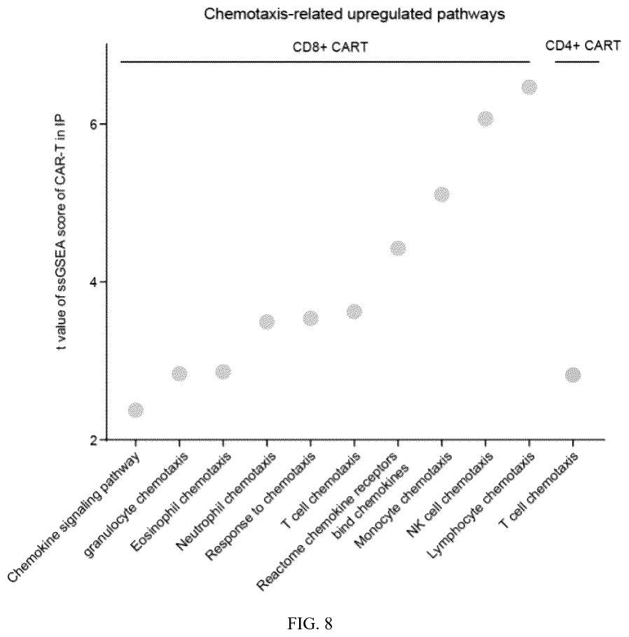

FIG. 9 shows the expression level of CXCR3 and the decline level of soluble B-cell maturation antigen (SBCMA) in cerebrospinal fluid.

### DETAILED DESCRIPTION OF THE EMBODIMENTS

The technical solutions of this disclosure will be clearly and completely described below with reference to the accompanying drawings. Obviously, the described embodiments are only some but not all of the embodiments of this disclosure. Based on the embodiments in this disclosure, all other embodiments obtained by those of ordinary skill in the art without making creative efforts shall fall within the scope of protection of this disclosure.

In the following embodiments, neuromyelitis optica spectrum disorder (NMOSD) was selected as a representative of central nervous system (CNS) diseases for testing. The neuromyelitis optica spectrum disorder (NMOSD) is an autoimmune inflammatory demyelinating disease of the central nervous system (CNS), and mainly manifests as optic neuritis and acute transverse myelitis related symptoms, such as visual deterioration, numbness and weakness of limbs, dyskinesia, and abnormal autonomic nervous function. The pathogenesis of NMOSD is mainly as follows: plasma cells produce aquaporin-4 (AQP4) antibodies, which bind to AQP4 antigens on astrocytes through a blood-brain barrier, and finally causes astrocyte damage, oligodendrocyte damage, demyelination, and neuron loss through mechanisms such as antibody dependent cytotoxicity and complement dependent cytotoxicity.

Subjects of clinical trials selected in the following specific embodiments included 5 patients with refractory or relapsed aquaporin-4 (AQP4) serum antibody-positive NMOSD treated with anti-BCMA CAR-T cells. Cerebrospinal fluid samples were obtained from the NMOSD patients separately at baseline and 3 months after infusion of the CAR-T cell.

Besides, 5 control subjects matched for age and gender with the NMOSD subjects were selected, including 1 patient with primary headache, 3 patients with non-inflammatory peripheral neuropathy, and 1 patient with idiopathic intracranial hypertension, from whom blood and cerebrospinal fluid samples were obtained separately. As approved by the Institutional Review Board of Tongji Hospital, the patients provided documented informed consent for the use of their infused products, blood, and cerebrospinal fluid samples in the trial related to the following embodiments.

In the following specific embodiments, cerebrospinal fluid samples were obtained from the NMOSD patients separately at baseline and 3 months after infusion of the CAR-T product.

The CAR-T cells provided and infused in the following embodiments were CAR-T cells targeting B-cell maturation antigens (BCMAs), which can highly express a chemokine receptor CXCR3 and chemokines CCL1, CCL3, and CCL4. These CAR-T cell products used lentivirus as a gene vector to transfect autologous T cells, and the CAR contains fully human scFv, CD8a hinge and transmembrane, 4-1BB costimulation, and CD3ζ activation domains. The CAR-T cell, with a research and development code of CT103A, was prepared by Nanjing IASO Biotherapeutics Co., Ltd.

Specifically, the method for obtaining the CAR-T cells provided in the following embodiments was as follows:
(1) First, the patients' leukocytes were separated using CD3 microbeads (Miltenyi Biotec) according to the manufacturer's protocol;
(2) The isolated leukocytes were then activated using Dynabeads™ Human T-Activator CD3/CD28 (Invitrogen) in the CTS™ OpTmizer™ culture medium (Gibco), together with 20 mM L-GlutaMAX™ (Gibco) and 200 IU/mL recombinant human IL-2 (SL PHARM);
(3) The CAR-T cells obtained in the step (2) used lentivirus as a gene vector to transfect autologous T cells, and the CAR contained fully human scFv, CD8a hinge and transmembrane, 4-1BB costimulation, and CD3ζ activation domains.

The transfected cells were washed and debeaded on D5 and then cultured in G-Rex (Wilson Wolf). On D10-D11, the CAR-T cells were collected, washed, and suspended in a cryopreservation solution; and
(4) Finally, the prepared CAR-T cells were aliquoted into freezing bags (Miltenyi Biotec) and frozen to −90° C. using ThermoFisher's 7451TF CRF #4. The final products were stored in a gas-phase liquid nitrogen tank (BIOBANK-22K) below −130° C.

### Test Example 1: Collection of CAR-T Cells Capable of Effectively Crossing the Blood-Brain Barrier

### 1. Sample Collection, Preparation, and Single-Cell RNA Sequencing

(1) Sterile centrifuge tubes were used to collect cerebrospinal fluid samples from the NMOSD patients at baseline and 1 month and 3 months after infusion, and also cerebrospinal fluid samples from the control subjects.
(2) The collected cerebrospinal fluid samples were centrifuged at 300×g for 10 minutes at 4° C. to obtain precipitated cells.
(3) The precipitated cells were resuspended in 60 μL of sterile phosphate buffer, and the single cell suspension was loaded onto the 10X Chromium platform for loading. Cell Ranger was used to demultiplex and align raw RNA data and antibody tag sequences.

The feature-barcode matrix generated from this pipeline was analyzed in R using the Seurat v4.2.0 software package for subsequent single-cell data analysis.

### 2. Detection of a Copy Number of CAR Genes in Cerebrospinal Fluid

The droplet digital PCR (ddPCR) technology can be used to detect the copy number of CAR genes in cerebrospinal fluid and evaluate the capability of CAR-T cells to cross the blood-brain barrier. This example used the ddPCR technology to absolutely quantify the copy number of CAR genes.

The specific method includes:
(1) Lysis buffer was added to the collected blood samples, sample DNA was then extracted using a blood genome DNA mini kit (51104, Qiagen) at a specified time point, and the CAR copy number of each sample was measured using the ddPCR;
(2) The copy number of CAR genes was determined using ddPCR. The probe and primer targeted scFV sequences. Specifically, the primer sequences of the CAR genes were as follows:

Forward primer: 5′-cagcaaaaatacgacctcctcact-3′; as shown in SEQ ID NO. 1;

Reverse primer: 5′-tggtgctgcctttgatctca-3′; as shown in SEQ ID NO. 2;

The primer sequences of the control group were as follows:

Forward primer: 5′-ggcggtggtcctggagtact-3′; as shown in SEQ ID NO. 3;

Reverse primer: 5′-agaggcctttggctttcttctt-3′; as shown in SEQ ID NO. 4;

The probe sequence was 5′-VIC-acccgccacaagc-3′, as shown in SEQ ID NO. 4 (with no fluorescent label); and
(3) The copy number was determined by quantitative real-time PCR with reference to a standard curve. A template-free control reaction was carried out using nuclease-free water.

The specific PCR process was as follows: performing one cycle at 50° C. for 2 min; performing one cycle at 95° C. for 10 min; performing 45 cycles at 95° C. for 15 s; performing one cycle at 60° C. for 60 s; and finally, kept at 4° C.

### 3. Detection of the Expression Levels of the Copy Number of CAR Genes in Cerebrospinal Fluid

In this disclosure, the expression levels of the copy number of CAR genes in cerebrospinal fluid from 5 NMOSD CAR-T subjects at baseline, 1 month and 3 months after infusion of the product were detected.

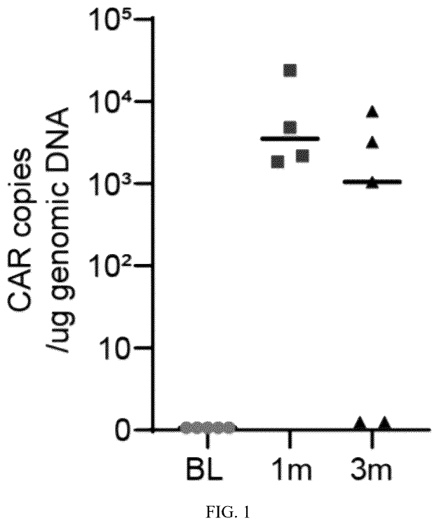

The detection results were shown in FIG. 1. In FIG. 1, Baseline represented the copy number of CAR genes in cerebrospinal fluid of the subjects at baseline, 1m represented the copy number of CAR genes in cerebrospinal fluid of the subjects 1 month after infusion of the product, and 3m represented the copy number of CAR genes in cerebrospinal fluid of the subjects 3 months after infusion of the product.

It may be seen that the expression levels of CAR genes in cerebrospinal fluid of the CAR-T subjects were significantly increased 1 month after infusion of the product, which indicated that the CAR-T cells through peripheral infusion crossed the blood-brain barrier to enter the cerebrospinal fluid at this time. However, 3 months after infusion of the product, the detected copy number suggested that the CAR-T cells had dropped to zero in cerebrospinal fluid of some patients, but maintained at a high level in cerebrospinal fluid of other patients.

The detection results in FIG. 1 showed that in cerebrospinal fluid of the central nervous system, the CAR-T cells with good blood-brain barrier permeability can maintain a high level of the copy number for a long time after infusion. This kind of CAR-T cells can effectively cross the blood-brain barrier, thus playing its key therapeutic effect in the central nervous system.

### Test Example 2: Determination of Highly Expressed Markers in CAR-T Cells Capable of Effectively Crossing the Blood-Brain Barrier

The R studio software was used to carry out analysis of single-cell differential genes, the differential genes between the CAR-T cells (CSF Exist) capable of effectively crossing the blood-brain barrier and the CAR-T cells (CSF noExist) not capable of effectively crossing the blood-brain barrier were analyzed and compared.

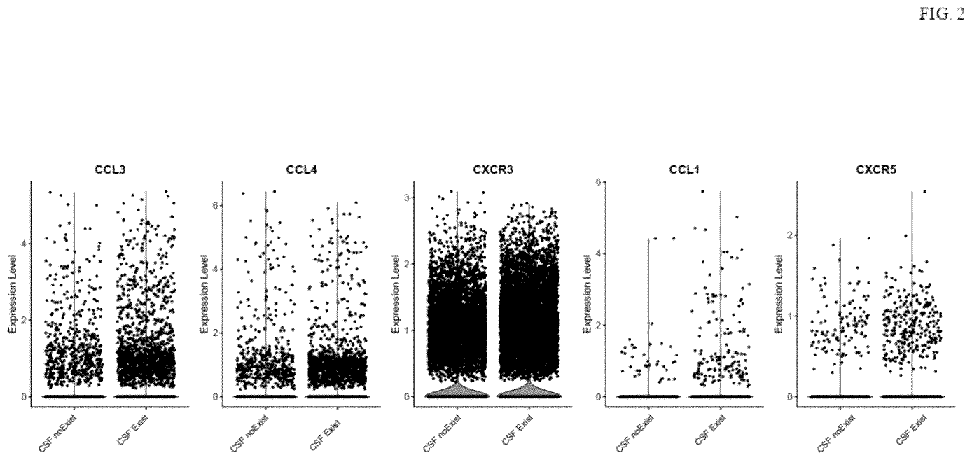

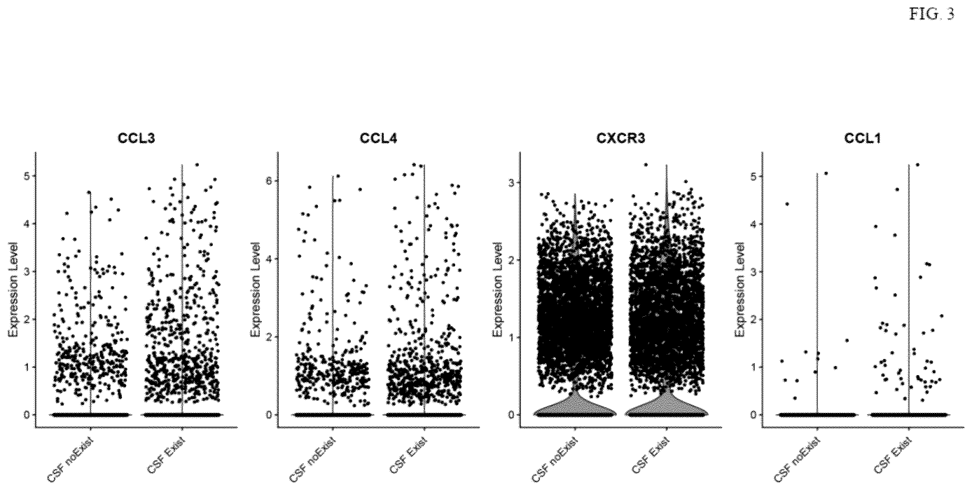

The analysis results were shown in FIG. 2 and FIG. 3. The results showed that:
(1) In FIG. 2, in CD4+ T cells, chemokine receptors CXCR3 and CXCR5 and chemokines CCL1, CCL3, and CCL4 in the CAR-T cells that still crossed the blood-brain barrier and reached the cerebrospinal fluid after three months of infusion were highly expressed; and
(2) In FIG. 3, in CD8+ T cells, the expression levels of the chemokine receptor CXCR3 and chemokines CCL1, CCL3, and CCL4 in the CAR-T cells that still crossed the blood-brain barrier and reached the cerebrospinal fluid after three months of infusion were significantly improved.

The results in FIG. 2 and FIG. 3 showed that the high expression of the chemokine receptor CXCR3 and chemokines CCL1, CCL3, and CCL4 can promote the CAR-T cells to cross the blood-brain barrier continuously, thus reaching the cerebrospinal fluid to exert their effects. Therefore, the chemokine receptor CXCR3 and chemokines CCL1, CCL3, and CCL4 can be used as biomarkers of CAR-T cells effectively crossing the blood-brain barrier, so as to develop a CAR-T cell with good blood-brain barrier permeability.

### Test Example 3: CAR-T Cells with Good Blood-Brain Barrier Permeability had Cell Chemotaxis and Highly Expressed Chemokines and Receptors Thereof

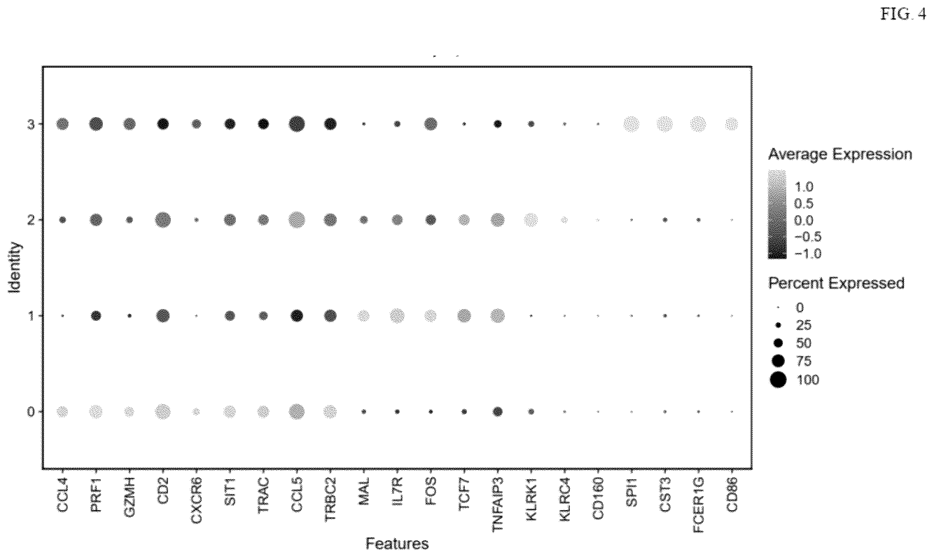

1. The single-cell RNA-seq was mapped to a 5′CAR sequence, a total of 287 CAR-T cells (that is, all CAR-T cells) were identified in this test example at 3 months, including 150 cells from 5 peripheral blood samples and 137 cells from 5 cerebrospinal fluid samples.
2. Single-cell RNA-seq data was re-clustered, four different clusters (0, 1, 2, and 3) were further identified according to the expression of markers from the CAR-T cells 3 months after infusion of the CAR-T product, as shown in FIG. 4, and the cells in peripheral blood and cerebrospinal fluid were distinguished.

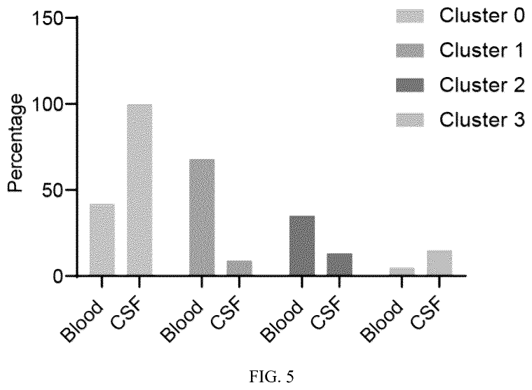

As shown in FIG. 5, Blood represented CAR-T cells in peripheral blood samples, CSF represented CAR-T cells in cerebrospinal fluid samples, and the vertical coordinate represented the percentage of each cluster. It was found from analyzing the characteristics of gene expression of different cell clusters that the proportion of the cell cluster 0 in cerebrospinal fluid was significantly higher than that of the blood cell. This showed that the cell cluster 0 can effectively cross the blood-brain barrier and enter the cerebrospinal fluid of the central nervous system.

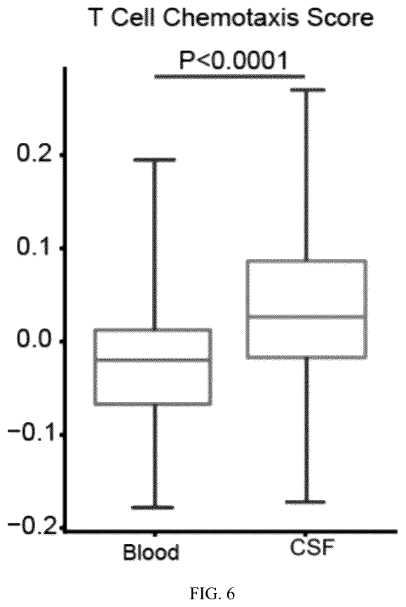

Further, the chemotaxis of the CAR-T cells in the peripheral blood was compared with that in the cerebrospinal fluid using the classical T cell chemotaxis scoring tool. The results were shown in FIG. 6, in which the vertical coordinate represented the chemotaxis score of T cells, the left side in the figure showed the chemotaxis score of CAR-T cells in peripheral blood samples, and the right side in the figure showed the chemotaxis score of CAR-T cells in cerebrospinal fluid samples. The results in FIG. 6 showed that the cells in cerebrospinal fluid had enhanced cell chemotaxis (P<0.0001). This showed that the CAR-T cells with high expression of the chemokines and receptors thereof can effectively cross the blood-brain barrier and enter the central nervous system.

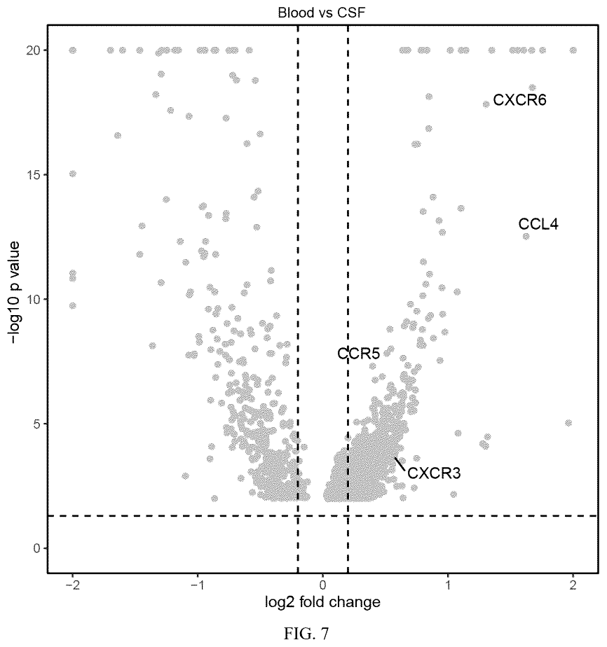

Based on the results of single-cell RNA sequencing analysis in the test example 1, the differential gene expression of cells in peripheral blood and cerebrospinal fluid was compared. As shown in FIG. 7, down-regulated differential genes were shown on the left side and up-regulated differential genes were shown on the right side through comparison. In the up-regulated differential genes, the expression levels of chemokine receptors CXCR3 and CCR5 in cerebrospinal fluid were significantly increased, and the expression levels of chemokines CCL4 and CXCL16 were also significantly increased. This showed that the CAR-T cells with high expression of the chemokines and receptors thereof can effectively cross the blood-brain barrier and enter the central nervous system.

Furthermore, the activation of chemotaxis-related pathways in CAR-T cells capable of effectively crossing the blood-brain barrier was analyzed by single-sample gene set enrichment analysis (GSEA). The analysis results were shown in FIG. 5, and it can be seen that both CD4+CAR-T cells and CD8+CAR-T cells, which effectively crossed the blood-brain barrier, were significantly up-regulated in the chemotaxis-related pathways. This showed that the CAR-T cells with good blood-brain barrier permeability can up-regulate the chemotaxis-related pathways, thus entering the central nervous system to play its targeted therapeutic effect.

### Test Example 4: Evaluation of Therapeutic Efficacy of CAR-T Cells with Good Blood-Brain Barrier Permeability in Central Nervous System

BCMA is a transmembrane glycoprotein in the tumor necrosis factor superfamily, and is mainly expressed on the surface of mature B lymphocytes. The soluble B-cell maturation antigen (sBCMA) is directly detached from the membrane BCMA, and is closely related to the injury of the central nervous system.

In this disclosure, human BCMA/TNFRSF17 ELISA kit (R&D Systems, DY193) was adopted to detect the expression levels of sBCMA in cerebrospinal fluid of subjects at baseline and after infusion of the CAR-T cell product, and the decline levels of sBCMA were calculated.

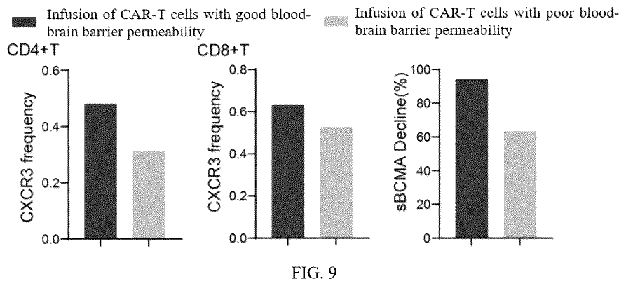

By comparing the expression levels of CXCR3 in CD4+ T cells and CD8+ T cells and the corresponding decline levels of sBCMA of the subjects, the results in FIG. 9 showed that the subjects after infusion of CAR-T cells with good blood-brain barrier permeability had higher expression levels of CXCR3, and the decline degree of sBCMA was higher, which indicated that the CAR-T cell product with good blood-brain barrier permeability could enhance the efficacy of CAR-T cells in the autoimmune of the central nervous system.

The above embodiments describe the implementation of this disclosure in detail, but this disclosure is not limited to the specific details in the above embodiments. Within the scope of the claims and technical concept of this disclosure, various simple modifications and changes may be made to the technical solutions of this disclosure, and these simple modifications and changes shall all fall within the protection scope of this disclosure.

SEQUENCE LISTING

SEQ ID NO. 1: Forward primer sequence of CAR gene
cagcaaaaatacgacctcctcact
SEQ ID NO. 2: Reverse primer sequence of CAR gene
tggtgctgcctttgatctca
SEQ ID NO. 3: Forward primer sequence of the
control group
ggcggtggtcctggagtact
SEQ ID NO. 4: Reverse primer sequence of the
control group
agaggcctttggctttcttctt
SEQ ID NO. 5: Probe sequence
acccgccacaagc

What is claimed is:

1. A CAR-T cell with good blood-brain barrier permeability, wherein the CAR-T cell is used for treating or improving central nervous system diseases, and the CAR-T cell targets B-cell maturation antigens and highly expresses a chemokine receptor CXCR3 and chemokines CCL1, CCL3, and CCL4.

2. The CAR-T cell with good blood-brain barrier permeability according to claim 1, wherein the CAR-T cell is derived from peripheral blood mononuclear cells (PBMCs) isolated from peripheral blood.

3. A method for detecting the blood-brain barrier permeability of the CAR-T cell according to claim 1 that is not aimed at disease detection and diagnosis, wherein judgment is made by detecting expression levels of the chemokine receptor CXCR3 and the chemokines CCL1, CCL3, and CCL4 in cerebrospinal fluid, or by detecting and analyzing chemotaxis of the CAR-T cell based on single-cell transcriptome sequencing data.

4. A product for detecting the blood-brain barrier permeability of the CAR-T cell, wherein the CAR-T cell is the CAR-T cell as claimed in claim 1, the CAR-T cell is used for treating or improving central nervous system diseases, and the product is used for detecting the expression levels of the chemokine receptor CXCR3 and the chemokines CCL1, CCL3, and CCL4 in cerebrospinal fluid.

5. The product according to claim 4, wherein the product comprises a reagent or probe for detecting the expression levels of the chemokine receptor CXCR3 and the chemokines CCL1, CCL3, and CCL4.

6. The product according to claim 4, wherein the product is a detection kit, a test strip, or a detection chip.

7. Use of the CAR-T cell according to claim 1, used for preparing drugs for targeted treatment or improvement of central nervous system diseases.

8. The use according to claim 7, wherein the central nervous system diseases are neuromyelitis optica spectrum disorders.


SEQUENCE LISTING (5 sequences)

SEQ ID NO: 1 - DNA, 24 bp, synthetic construct
  source 1..24: mol_type = other DNA
cagcaaaaatacgacctcctcact

SEQ ID NO: 2 - DNA, 20 bp, synthetic construct
  source 1..20: mol_type = other DNA
tggtgctgcctttgatctca

SEQ ID NO: 3 - DNA, 20 bp, synthetic construct
  source 1..20: mol_type = other DNA
ggcggtggtcctggagtact

SEQ ID NO: 4 - DNA, 22 bp, synthetic construct
  source 1..22: mol_type = other DNA
agaggcctttggctttcttctt

SEQ ID NO: 5 - DNA, 13 bp, synthetic construct
  source 1..13: mol_type = other DNA
acccgccacaagc